(19) United States
(12) Patent Application Publication
(10) Pub. No.: US 2009/0118775 A1
(43) Pub. Date: May 7, 2009

(54) Sternal Clamp

(76) Inventor: Shawn M. Burke, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/290,521

(22) Filed: Oct. 31, 2008

Related U.S. Application Data
(60) Provisional application No. 61/001,390, filed on Nov. 1, 2007.

(51) Int. Cl. A61B 17/04

(52) U.S. Cl. 606/324

(57) ABSTRACT

A sternal closure clamp for securing and retaining longitudinally divided halves of a sternum, the clamp having a pair of body members each having a pair of engagement members, a pair of guide beam members affixed to a first of the body members and extending through a second of the body members, the guide beam members having an I-beam cross-sectional configuration, and a threaded rod member retained by the first body member and passing through the second body member, whereby rotation of the rod member in a first direction increases the distance between the body members and rotation in a second direction decreases the distance between the body members.

This patent application claims the benefit of U.S. Provisional Patent Application Ser. No. 61/001,390, filed Nov. 1, 2007.

### BACKGROUND OF THE INVENTION

This invention generally relates to devices used to rejoin a human sternum that has been severed longitudinally, and more particularly relates to such devices that function in a clamping manner to retain the severed sternum portions in a closed and abutting relationship post-operatively.

It is often necessary in surgical operations to longitudinally sever the patient's sternum so that the ribs may be spread to provide access to internal organs such as the heart. It is then necessary to secure the sternum halves together for post-operative recovery. Various closure techniques are used to accomplish this task. For example, holes may be drilled into the sternum halves and suture material passed through and tightened to cinch the sternum halves together. Apertured plates may be added to further rigidify the sternum post-operatively, with the suture material being passed through the apertures in the plate and the sternum. Encircling members may be wrapped around the sternum and tightened. Toothed bridging members extending across the cut line may be pressed into the sternum surfaces and/or secured with threaded rods extending between the sternal halves.

Another sternal closure technique involves the use of clamps having hook-like projections or engagement members on both ends, the clamp being positioned laterally relative to the sternal incision with the projections being disposed between adjoining rib pairs. The clamp is then linearly contracted or compressed to shorten the device and force the sternal halves together, the clamp typically comprising two members joined in a linear telescoping manner. Locking or securing means, either permanent or releasable, maintain the clamp in the contracted configuration. Because high strength and rigidity are crucial characteristics of the sternal clamp, the main bodies of the clamps are often over-engineered in terms of thickness and mass.

It is also desirable to provide a mechanism whereby the clamp can be quickly and easily removed from the sternum if necessary, such as for example if the heart needs to be re-accessed after the sternal clamp has been applied. Known mechanisms for accomplishing this are often excessively complicated or result in loose components that can be dropped and lost within the chest cavity.

Examples of such techniques and devices are described in U.S. Pat. No. 3,473,528 to Mishkin et al., U.S. Pat. No. 4,201,215 to Crossett et al., U.S. Pat. No. 4,279,248 to Gabbay, U.S. Pat. No. 4,583,541 to Barry, U.S. Pat. No. 5,139,498 to Astudillo Ley, U.S. Pat. No. 6,051,007 to Hogendijk et al., U.S. Pat. No. 6,217,580 to Levin, U.S. Pat. No. 6,302,899 to Johnson et al., U.S. Pat. No. 6,540,769 to Miller, III, and U.S. Pat. No. 6,712,821 to Gabbay.

It is an object of this invention to provide a sternal closure clamp device where the sternal halves may be quickly and easily contracted and retained in position, or may be quickly expanded should it be necessary to reopen the sternum, and which provides greater reinforcement and rigidity to the severed sternum. It is a further object of this invention to provide such a sternal clamp that possesses high strength and rigidity but with reduced mass.

### SUMMARY OF THE INVENTION

The invention is in general a sternal closure clamp device for post-operatively closing, securing and supporting a patient's sternum that has been longitudinally severed into two sternal halves. The sternal clamp generally comprises two body members each having a pair of spaced, sternum-engaging engagement members extending from the body member. A pair of parallel guide beam members is affixed to a first body member, the guide beam members preferably being disposed to either side of a threaded rod member, which is retained within the first body member in a manner that allows the rod member to be rotated relative to the first body member, such as by passage through an unthreaded bore. The guide beam members are of a cross-sectional I-beam configuration in order to increase the overall rigidity of the device while reducing the overall mass. A drive member is provided on the outer end of the rod member to receive a drive tool for rotating the rod member.

The second body member is provided with a threaded bore that receives the turn screw member, and is further provided with a pair of guide beam apertures sized to receive the guide beam members in a manner that allows the second body member to move longitudinally relative to the guide beam members. With this construction, rotation of the threaded rod member in a first direction increases the distance between the body members, while rotation of the turn screw member in the second direction decreases the distance between the body members. Additional means to lock or fix the rod member in a non-rotatable status such that the separation distance between the body members may be fixed and secured when the body members are fully contracted about the sternal halves may also be present.

The engagement members are means to engage, secure or otherwise retain the sternal halves in an abutting relationship, with the engagement members comprising hooks, projections, fingers or the like extending in the posterior direction from the body members, whereby the engagement members can be disposed against the outer edges of the sternal halves and between adjoining ribs, preferably with the two engagement members located on a given body member being positioned in the inter-rib spaces to either side of a single rib. Preferably the engagement members are generally L-shaped, having defined leg members and foot members, the foot members extending inwardly approximately at 90 degrees to the leg members and generally parallel to the guide beam members. Preferably the foot members are configured and aligned such that they may overlap rather than striking each other end-to-end when the two body members are brought together.

### BRIEF DESCRIPTION OF THE DRAWINGS

FIG. 1 is a perspective view of an embodiment of the invention.

FIG. 2 is a bottom view of the embodiment of FIG. 1, showing the overlapping alignment of the foot members.

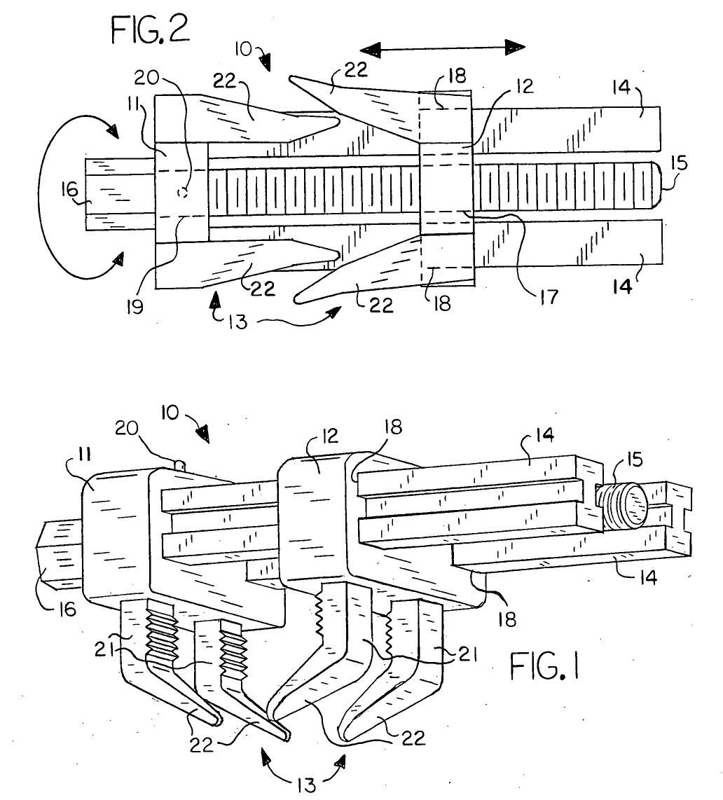

### DETAILED DESCRIPTION OF THE INVENTION

With reference to the drawings, the invention will now be described in detail with regard for the best mode and the preferred embodiment. The invention is a sternal closure clamp device used to close, secure and support a sternum post-operatively, the sternum having been severed longitudinally into left and right lateral sternal halves to provide access to the interior of the chest.

The sternal clamp 10 generally comprises two opposing body members, a first body member 11 and a second body member 12, each having connected thereto a pair of spaced, sternum-engaging, engagement members 13 extending posteriorly from the body members 11 and 12. A pair of parallel guide beam members 14 is affixed to the first body member 11, the guide beam members 14 being disposed to either side of a threaded rod member 15, which is retained within a non-threaded centralized bore 19 in the first body member 11 in a manner that allows the rod member 15 to be rotated relative to the first body member 11. The guide beam members 14 are of an I-beam configuration in cross-section in order to increase the overall rigidity of the clamp 10 while reducing mass. A handle or drive member 16, such as a polygonal head, is provided on the outer end of the rod member 15 to optionally receive a drive tool (not shown) for rotating the rod member 15 within the first body member 11.

The second body member 12 is provided with an internally threaded bore 17 that receives the rod member 15 in cooperating manner, such that the threading of the bore 17 mates with the threading of the rod member 15 and rotation of the threaded rod member 15 results in axial movement of the second body member 12 relative to the rod member 15 and to first body member 11. The second body member 12 is further provided with a pair of guide beam passages 18 sized and configured to receive the guide beam members 14 in a manner that allows the second body member 12 to move in a controlled path longitudinally relative to the guide beam members 14. With this construction, rotation of the threaded rod member 15 in a first direction increases the distance between the body members 11 and 12, while rotation of the rod member 15 in the second and opposite direction decreases the distance between the body members 11 and 12. Friction may be sufficient to prevent undesired rotation of the turn screw member 15, but preferably mechanical means for locking the body members 11 and 12 in a fixed relative position once they are fully contracted about the sternal halves is provided, such as a locking pin or set screw 20 inserted into a bore provided in the first body member 11. To fix the position of the body members 11 and 12 once they are compressed against the sternal halves, the setscrew 20 is tightened against the rod member 15. To release the sternal clamp 10, the setscrew 20 is loosened so that that the rod member 15 is free to be rotated in the release direction.

The engagement members 13 are means to engage, secure or otherwise retain the sternal halves in an abutting relationship, with the engagement members 13 comprising hooks, projections, fingers or the like extending in the posterior direction from the main bodies 11 and 12, whereby the engagement members 13 are able to be disposed against the outer edges of the sternal halves and between adjoining ribs, preferably with the two engagement members 13 located on a given side being positioned in the inter-rib spaces to either side of a single rib. Preferably the engagement members 13 are generally L-shaped, having defined leg members 21 and foot members 22, the foot members 22 extending inwardly with each pair of foot members 22 on a given body member being directed toward the other pair of foot members 22, and the foot members 22 being disposed generally parallel to the guide beam members 14. Preferably, as shown in FIG. 3, the foot members 22 on the two body members are configured and aligned such that they may overlap or pass, such as in a side-to-side manner, rather than abutting or interfering with each other when the two body members 11 and 12 are brought closely together.

In use, the clamp 10 is positioned between the sternal halves across the longitudinal sternal incision with the engagement members 13 located in the inter-rib spaces, such that at least one rib is between the two engagement members 13 on a given side of the clamp 10. The sternal halves are drawn together to close the longitudinal incision either directly by rotation of the drive member 16 and rod member 15 of the sternal clamp 10, or by using other instruments such as forceps to press the sternal halves together and then tightening the clamp 10 by rotation of the rod member 15. In this manner the sternal halves are retained in an abutted relation by the engagement members 13 and are precluded from separating during the healing process.

In the event that the sternal halves do need to be separated after the device has been put into position, such as to address a post-operative problem or emergency, the body members 11 and 12 are separated by turning the drive member 16 and turn screw member 15 in the opposite direction, thus allowing the clamp 10 to be removed and the sternal halves opened. In similar manner that clamp 10 can be removed from the patient if desired after the sternum has healed.

It is contemplated that equivalents and substitutions for certain elements set froth descriptively above may be obvious to those of ordinary skill in the art, and therefore the true scope and definition of the invention is to be asset forth in the following claims.

I claim:

1. A sternal closure clamp comprising:
a first body member and a second body member, each said body member having a pair of engagement members connected thereto, said engagement members each comprising a leg member and foot member;
an internally threaded bore disposed in said second body member;
a pair of parallel guide beam members affixed to said first body member and extending through said second body member, said guide beam members having an I-beam cross-sectional configuration; and
a threaded rod member retained by said first body member in a rotatable manner and passing through said threaded bore of said second body member, whereby rotation of said rod member in a first direction increases the distance between said body members and rotation in a second direction decreases the distance between said body members.

2. The clamp of claim 1, further comprising means for locking said first and second body members in a fixed relative position.

3. The clamp of claim 2, wherein said means for locking said first and second body members in a fixed relative position comprises a set screw positioned in said first body member, such that said set screw abuts said rod member.

4. The clamp of claim 1, wherein said foot members of said first body member and said foot members of said second body member are configured and aligned such that the foot members do not abut when said first and second body members are brought closely together.

5. The clamp of claim 2, wherein said foot members of said first body member and said foot members of said second body member are configured and aligned such that the foot members do not abut when said first and second body members are brought closely together.

6. The clamp of claim 3, wherein said foot members of said first body member and said foot members of said second body member are configured and aligned such that the foot members do not abut when said first and second body members are brought closely together.

7. A sternal closure clamp comprising:
a first body member and a second body member, each said body member having a pair of engagement members connected thereto, said engagement members each comprising a leg member and foot member, wherein said foot members of said first body member are directed toward said foot members of said second body member and said foot members of said second body member are directed toward said foot members of said first body member;
an internally threaded bore disposed in said second body member;
a pair of parallel guide beam members affixed to said first body member and extending through said second body member, said guide beam members having an I-beam cross-sectional configuration; and
a threaded rod member retained by said first body member in a freely rotatable manner and passing through said threaded bore of said second body member, said rod member being parallel to said guide members, said threaded bore of said second body member and said threaded rod cooperating in a mated manner whereby rotation of said rod member causes axial movement of said second body member relative to said first body member and said guide beam members.

8. The clamp of claim 7, further comprising means for locking said first and second body members in a fixed relative position.

9. The clamp of claim 8, wherein said means for locking said first and second body members in a fixed relative position comprises a set screw positioned in said first body member, such that said set screw abuts said rod member.

10. The clamp of claim 7, wherein said foot members of said first body member and said foot members of said second body member are configured and aligned such that the foot members do not abut when said first and second body members are brought closely together.

11. The clamp of claim 8, wherein said foot members of said first body member and said foot members of said second body member are configured and aligned such that the foot members do not abut when said first and second body members are brought closely together.

12. The clamp of claim 9, wherein said foot members of said first body member and said foot members of said second body member are configured and aligned such that the foot members do not abut when said first and second body members are brought closely together.
